(19) United States
(12) Patent Application Publication
(10) Pub. No.: US 2007/0060776 A1
(43) Pub. Date: Mar. 15, 2007

(54) Method for the production of alpha-(3-arylthio)-acetophenones

(75) Inventors: Marco Altmayer, IIvesheim (DE); Wolfgang Siegel, Limburgerhof (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 10/575,539

(22) PCT Filed: Oct. 14, 2004
(86) PCT No.: PCT/EP04/11521

§ 371 Date: Apr. 12, 2006

(30) Foreign Application Priority Data
Oct. 20, 2003 (DE) 103 49 249.6

(51) Int. Cl. C07C 323/22

(52) U.S. Cl. 568/43

(57) ABSTRACT

A process for preparing α-(3-arylthio)acetophenones of the general formula I 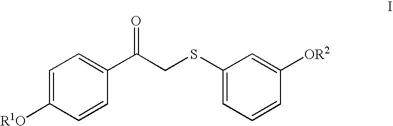
in which the substituents R¹ and R² are each independently C₁-C₆-alkyl, SiR³₃ where the substituent R³ is a C₁-C₆-alkyl radical, or an optionally substituted phenyl or benzyl radical, which comprises reacting, in methanol acetophenones of the general formula II 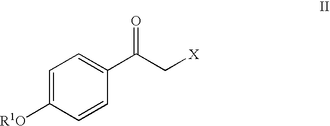
in which the substituent X is Cl or Br with a thiolate of the general formula III 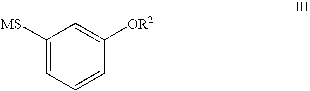 in which M is an alkali metal.

The present invention relates to an improved process for preparing α-(3-arylthio)-acetophenones of the general formula I 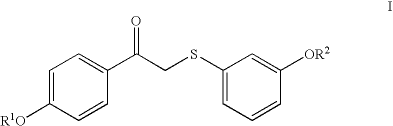
in which the substituents R¹ and R² are each independently C₁-C₆-alkyl, SiR³₃ where the substituent R³ is a C₁-C₆-alkyl radical, or an optionally substituted phenyl or benzyl radical.

The compounds of the formula I are valuable intermediates in the synthesis of pharmaceutically active substances; 1-(4-methoxyphenyl)-2-[(3-methoxyphenyl)thio]ethanone (R¹, R²=methyl) is a building block for the preparation of the active antiosteoporosis ingredient Raloxifen.

Various methods are known for preparing acetophenones of the general formula I. Starting compounds are in most cases the acetophenones of the general formula II 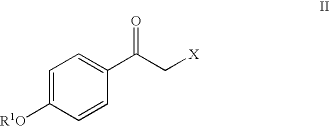
in which the substituent X is chlorine or bromine and the substituent R¹ is as defined above. These compounds are reacted with a thiol
in a biphasic system composed of ethyl acetate and potassium hydroxide solution (WO 02/42261)
in an ethanol/water/ethyl acetate mixture with potassium hydroxide (Tetrahedron Letters 40 (1999) 2909)
in ethanol with potassium hydroxide solution (U.S. Pat. No. 4,133,814)
in an ethanol/water mixture with potassium hydroxide solution (U.S. Pat. No. 4,418,068).

The maximum yield for the particularly sought-after 1-(4-methoxyphenyl)-2-[(3-methoxyphenyl)thio]ethanone by these methods is 86%.

It is an object of the present invention to provide a process which enables a higher yield of product of value.

We have found that this object is achieved by reacting, in methanol acetophenones of the general formula II 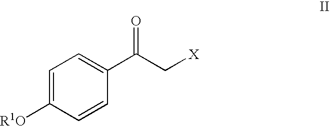
in which the substituent X is Cl or Br and the substituent R¹ is C₁-C₆-alkyl, SiR³₃ where the substituent R³ is a C₁-C₆-alkyl radical, or an optionally substituted phenyl or benzyl radical, with a thiolate of the general formula III 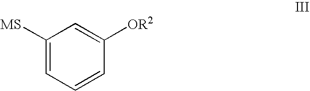
in which M is an alkali metal.

The process according to the invention serves to prepare compounds of the general formula I, preferably 1-(4-methoxyphenyl)-2-[(3-methoxyphenyl)thio]ethanone.

One starting compound is a chloro- or bromoacetophenone of the general formula II in which the substituent R¹ is C₁-C₆-alkyl such as methyl, ethyl, isopropyl, n-butyl or iso-butyl, phenyl or benzyl, in which case the phenyl or benzyl radicals may bear substituents which are inert under the reaction conditions, for example halogen or oxyalkyl, or the substituent R¹ is tri(C₁-C₆)alkylsilyl groups, preferably trimethylsilyl. R¹ is preferably a short-chain alkyl radical, in particular methyl. These compounds are obtainable in a manner known per se, for example by reacting acetophenones with sulfuryl chloride (U.S. Pat. No. 5,710,341) or with bromine (Chem. Ber. 1953, 86, 1556).

The acetophenones of the general formula II are reacted with a thiolate of the general formula III in which the substituent R² is C₁-C₆-alkyl such as methyl, ethyl, isopropyl, n-butyl or isobutyl, phenyl or benzyl, in which case the phenyl or benzyl radicals may bear substituents which are inert under the reaction conditions, for example halogen or oxyalkyl, or the substituent R² is tri(C₁-C₆)alkylsilyl groups, preferably trimethylsilyl. R² is preferably a short-chain alkyl radical, in particular methyl.

The thiolate cation M is an alkali metal such as lithium, sodium or potassium. The thiolates may be prepared by deprotonating the corresponding thiols. To this end, the thiols are reacted with a base whose base strength is sufficient to deprotonate the thiol. This may be effected in a separate reaction with isolation of the thiolate, although preference is given to in situ preparation of the thiolate and subsequent conversion to acetophenones of the general formula I. Preferred bases for the in situ preparation of the thiolates are alkali metal hydroxides such as potassium hydroxide and sodium hydroxide, hydrides such as lithium hydride and sodium hydride, amides such as lithium amide, sodium amide and potassium amide and alkoxides such as sodium methoxide and potassium methoxide. Particular preference is given to sodium methoxide.

The reaction of the chloro- or bromoacetophenones of the general formula II with a thiolate of the general formula III proceeds in methanol. The methanol may also contain small amounts of further polar solvents such as water, but preferably not more than 5% by weight thereof.

The molar ratios of the starting compounds are generally from 0.8 to 2.0 mol of thiolate of the general formula III per mole of the chloro- or bromoacetophenone of the general formula II, preferably from 0.90 to 1.05 mol per mole.

The reaction may be undertaken, for example, in a stirred tank. Preference is given to initially charging the chloro- or bromoacetophenone of the general formula II in methanol.

The amount of methanol is generally 100-1000 g, based on 100 g of the acetophenone of the general formula II used, preferably 150-200 g. To this end, preference is given to metering the thiolate of the general formula III into methanol, using 100-1000 g of methanol, preferably 150-200 g, for 100 g of thiophenol used.

The reaction may be carried out at atmospheric pressure and a temperature of preferably from 0 to 50° C. The end of the reaction may be detected, for example, by gas chromatography.

The sought-after products of value of the general formula I are only sparingly soluble in methanol and are therefore obtained as a solid in the reaction. They can be isolated in a simple manner by filtration. The alkali metal chloride or bromide which is formed and precipitates in the reaction can be removed readily by washing with water.

The process according to the invention allows the preparation of compounds of the general formula I in high yield and can additionally be carried out in a simple manner from a process technology point of view.

### EXAMPLE 1

### Preparation of 1-(4-methoxyphenyl)-2-[(3-methoxyphenyl)thio]ethanone

216 g (1.54 mol) of 3-methoxythiophenol were initially charged in 253 g (320 ml) of methanol in a 2 l stirred apparatus. At a maximum temperature of 35° C., 275 g (1.51 mol) of a 30% methanolic sodium methoxide solution were added dropwise. Afterward, a further 127 g (160 ml) of methanol were added to the mixture.

The above-described 3-methoxythiophenolate solution was added dropwise at a maximum of 35° C. to 285 g (1.54 mol) of chloromethoxyacetophenone in 494 g (624 ml) of methanol in a 5 l stirred flask. The mixture was stirred at ambient temperature for 10 minutes and then at 0° C. for 1 h. The crystals were filtered off with suction, washed with 1.5 l of water to free them of salts and then washed with 928 ml of methanol. The colorless product was dried at 30° C. under reduced pressure.

Yield: 424 g (1.47 mol): 97.4% with a purity of 99.3% (GC area %)

1. A process for preparing α-(3-arylthio)acetophenones of the general formula I 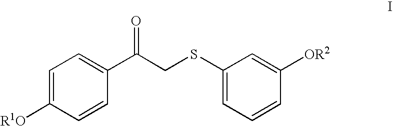
in which the substituents R¹ and R² are each independently C₁-C₆-alkyl, SiR³₃ where the substituent R³ is a C₁-C₆-alkyl radical, or an optionally substituted phenyl or benzyl radical, which comprises reacting, in methanol acetophenones of the general formula II 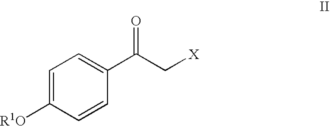
in which the substituent X is Cl or Br with a thiolate of the general formula III 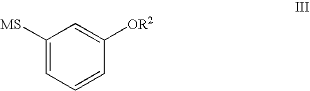
in which M is an alkali metal.

2. A process as claimed in claim 1, wherein 1-(4-methoxyphenyl)-2-[(3-methoxyphenyl)thio]ethanone is prepared.

3. A process as claimed in claim 1, wherein M is sodium.

4. A process as claimed in claim 1, wherein a thiolate of the general formula III is prepared by reacting the appropriate thiol with sodium methoxide.

5. A process as claimed in claim 2, wherein M is sodium.

6. A process as claimed in 5, wherein a thiolate of the general formula III is prepared by reacting the thiol with sodium methoxide.
